(19) United States
(12) Patent Application Publication
(10) Pub. No.: US 2023/0091676 A1
(43) Pub. Date: Mar. 23, 2023

(54) GROUPING SIMILAR EPISODES DETECTED BY A WEARABLE MEDICAL DEVICE (WMD)

(71) Applicant: West Affum Holdings Corp., Grang Cayman (KY)

(72) Inventors: Jaeho Kim, Redmond, WA (US); Joseph L. Sullivan, Kirkland, WA (US); David P. Finch, Bothell, WA (US)

(21) Appl. No.: 17/952,177

(22) Filed: Sep. 23, 2022

Related U.S. Application Data
(60) Provisional application No. 63/247,706, filed on Sep. 23, 2021.

(51) Int. Cl. A61B 5/282; A61B 5/364; A61B 5/333; A61B 5/339; A61B 5/00; A61N 1/39; A61B 5/366

(52) U.S. Cl. CPC A61B 5/282; A61B 5/364; A61B 5/333; A61B 5/339; A61B 5/0006; A61N 1/3904; A61B 5/366; A61B 5/0022; A61B 5/6802

(57) ABSTRACT

Technologies and implementations related to facilitating grouping of electrocardiogram (ECG) signals of a heart of a person (e.g., patient) wearing a wearable medical device (WMD). The ECG signals may be acquired during various times (e.g., during a normal rhythm of the heart and/or during an event of the rhythm of the heart) including various times of activity of the person (e.g., sleeping, awake, active, inactive, etc.). The ECG signals may be received and analyzed to determine if the ECG signals may be indicative of an event associated with a heart of the person or not.

### RELATED APPLICATION

This application claims benefit of priority to U.S. Provisional Patent Application Ser. No. 63/247,706, filed on Sep. 23, 2021, titled GROUPING SIMILAR EPISODES DETECTED BY A WMD, which is incorporated herein by reference in its entirety for all purposes.

### INFORMATION

Unless otherwise indicated herein, the approaches described in this section are not prior art to the claims in this application and are not admitted to be prior art by inclusion in this section.

Technology has contributed to improvements in healthcare. Some examples include healthcare related devices that may be capable of determining various health related information about a person. For example, a healthcare device may be capable of determining health related information of an electrical activity of a heart of a person.

The healthcare device may be included as part of a wearable system, where the wearable system may include a healthcare device configured to be worn by the person (e.g., a wearable medical device or a WMD) facilitating a more continuous monitoring and/or treatment of various health related issues of the person. For example, the WMD may be configured to monitor the electrical activities and/or treat potential health related issues of the heart. The electrical activities of the heart may be received by the WMD via one or more electrodes, where the one or more electrodes may be configured to receive electrical signals from the heart and communicate the electrical signals to the WMD.

The wearable system having the WMD may receive and store a variety of information about the health of the person and any treatment that may have been administered to the person by the WMD. The variety of information may be informative to various people that may interact with the person (e.g., first responder, medical personnel, medical professional, etc.). For example, the electrical activities of the heart may be captured by the WMD in the form of electrocardiogram (ECG) data. The ECG data may be of informative to a medical professional (e.g., a doctor), where the person may be a patient of the doctor.

The variety of information may include information that may not be informative (i.e., information that may not be helpful for the medical professional) such as, but not limited to, electrical noise, electrical artifacts, issues with the functionality of the WMD, some form of activity of the patient, etc. Unfortunately, the not so informative information may be processed and stored, which may cause the medical professional to evaluate a large volume of information to discern what information is helpful to the treatment of the patient and what information may not be helpful to the treatment of the patient.

All subject matter discussed in this section of this document is not necessarily prior art and may not be presumed to be prior art simply because it is presented in this section. Plus, any reference to any prior art in this description is not and should not be taken as an acknowledgement or any form of suggestion that such prior art forms parts of the common general knowledge in any art in any country. Along these lines, any recognition of problems in the prior art are discussed in this section or associated with such subject matter should not be treated as prior art, unless expressly stated to be prior art. Rather, the discussion of any subject matter in this section should be treated as part of the approach taken towards the particular problem by the inventor(s). This approach in and of itself may also be inventive. Accordingly, the foregoing summary is illustrative only and not intended to be in any way limiting. In addition to the illustrative aspects, embodiments, and features described above, further aspects, embodiments, and features will become apparent by reference to the drawings and the following detailed description.

### SUMMARY

Described herein are various illustrative wearable medical device (WMD) systems and methods for configurable comparisons of portions of electrocardiogram (ECG) data. Example WMD systems may include a configurable comparator module (CCM) that may be configured to capture a portion of an ECG data. In some examples, the CCM may be configured to capture the first portion of the ECG data based on a predetermined parameter. Example WMD systems may include the CCM configured to capture a second portion of the ECG data. The CCM may be configured to cause to display the first portion and the second portion concurrently for comparison by a user.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a block diagram of a system for grouping ECG signals in accordance with various embodiments.

FIG. 2 illustrates an example of a medical device that may be utilized, in accordance with at least one or more example embodiments.

FIG. 3 illustrates an example of an output of various signals from a WMD in accordance with various embodiments.

FIG. 4 shows part A of the sample output 300 (shown in FIG. 3) in further detail.

FIG. 5 shows part B of the sample output 300 (shown in FIG. 3) in further detail.

FIGS. 6A-6C illustrate an example of an accelerometer configuration, which may be utilized with various embodiments.

FIG. 7 is a portion of a sample output showing some further detail in accordance with various embodiments.

FIGS. 8A and 8B illustrate some examples of determining a posture of a person in accordance with various embodiments.

FIG. 9 illustrates various signals, which may be related to an activity of a person in accordance with various embodiments.

FIG. 10 illustrates signals received from a person, who may be in a moving vehicle in accordance with various embodiments.

FIG. 11 illustrates signals related to potential issues with one or more ECG electrodes.

FIG. 12 illustrates signals related to HR and QRS signals in accordance with various embodiments.

FIG. 13 illustrates QRS widths signals in more detail.

FIG. 14 illustrates ECG signals of an episode that may be utilized to derive the charts of FIGS. 12 and 13.

FIG. 15 illustrates an operational flow for grouping of electrocardiogram (ECG) signals of a heart of a person (e.g., patient) wearing a wearable medical device (WMD) in accordance with various embodiments as described herein.

FIG. 16 illustrates an example computer program product, arranged in accordance with at least some embodiments described herein.

FIG. 17 is a block diagram illustrating an example computing device 1700, such as might be embodied by a person skilled in the art, which is arranged in accordance with at least some embodiments of the present disclosure.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to methods, apparatus, and wearable medical device (WMD) systems related to facilitating grouping of electrocardiogram (ECG) signals of a heart of a person (e.g., patient) wearing a wearable medical device (WMD). The ECG signals may be acquired during various times (e.g., during a normal rhythm of the heart and/or during an event of the rhythm of the heart) including various times of activity of the person (e.g., sleeping, awake, active, inactive, etc.). The ECG signals may be received and analyzed to determine if the ECG signals may be indicative of an event associated with a heart of the person. The ECG signals indicative of an event associated with the heart may be flagged as an episode related to the activity of the heart (i.e., ECG signals indicative of an episode related to the heart). The flagged episodes may be stored. An ECG grouping module (EGM) may analyze the episodes to determine if the episodes meet a pattern matching criterion. If any of the episodes meet a pattern matching criterion, these episodes may be grouped. The grouped episodes may be displayed to facilitate review by a medically trained person (e.g., a clinician). As a result, a burden on the clinician in reviewing episode data may be reduced by enabling the clinician to review one of the episodes of a group of similar episodes and apply this assessment to the rest of the episodes in the group of similar episodes.

For the purposes of providing a detailed description of the claimed subject matter, utilization of healthcare device may include a WMD and may be described as included in the WMD systems of the present disclosure. However, in various embodiments, the WMD systems of the present disclosure may include a variety of wearable devices such as, but not limited to, cardiac event monitors, Holter monitors, mobile cardiac telemetry (MCT) devices, brain activity monitors, wearable cardioverter defibrillators (WCDs), mobile devices (e.g., a mobile/smart phones), and so forth. Accordingly, the claimed subject matter is not limited in this respect.

Utilizing the example of the WMD system including the WMD, the WMD may be configured to facilitate monitoring of electrical signals such as, but not limited to, monitoring of electrical signals from a heart of a person. For example, the WMD may be configured to monitor potential issues with the heart (i.e., the patient may have a health condition, where the electrical control system of the heart may malfunction causing the heart to beat irregularly or not at all). In some examples, these types of WMDs may include a defibrillator device to treat potential issues with the heart. An example of a WMD, which may be configured to monitor and treat potential issues with the heart, may include a wearable cardioverter defibrillator (WCD). In the present disclosure, for the purposes of ease of understanding the various embodiments of the claimed subject matter, references may be made to a medical device such as, but not limited to, a WCD. However, as it is clearly described herein, the claimed subject matter may include a variety of electrical monitoring devices and/or systems.

As part of the description of utilization of the WCD, some issues with the heart may be briefly described. For example, some issues with the rate of the heartbeat may be generally referred to as an arrhythmia. Arrhythmia may be caused by many factors, but in general, an arrhythmia may be caused by a malfunction in the electrical control system of the heart. Some types of arrhythmias may result in inadequate blood flow resulting in reduction or lack of the amount of blood pumped to the various parts of the body. For example, issues with the sinoatrial (SA) node may lead to arrhythmia of some kind. Some arrhythmias may lead to a condition known as sudden cardiac arrest (SCA). In an SCA condition, the heart may fail to pump blood effectively, and as a result, death may occur.

An example type of an arrhythmia, which may be associated with SCA, may be a condition known as ventricular fibrillation (VF). VF may be a condition where a ventricle or ventricles, which make up the heart to facilitate the pumping of blood, may make uncoordinated movements instead of steady rhythmic movements. In the VF condition, the heart may not pump adequate amounts of blood or may not pump blood at all, which may eventually lead to death. Another type of arrhythmia, which may be associated with SCA, may be a condition known as ventricular tachycardia (VT).

Turning back to the WCD, the WCD may be capable of monitoring the electrical signals of the heart and if necessary (e.g., a heart rhythm event), administer therapy to the heart in the form of an electric shock. The WCD may monitor the electrical signals and provide the electric shock to the heart externally (i.e., through the surface of a body) via components commonly known as electrodes, where some of the electrodes may be monitoring electrodes and some of the electrodes may be therapy electrodes. The WCD may be included in a support structure configured to be worn by the person. In this example, the WCD may help facilitate monitoring the electrical activities of the heart and provide the electric shock to the heart in the VF condition. As a result, the medical device may help prevent Sudden Cardiac Death (SCD).

Before turning to the figures, non-limiting example scenarios of utilization of various embodiments of WMD system may be described. In one non-limiting example scenario, a patient may have a heart condition, where the patient may utilize a healthcare device (e.g., the WCD). As mentioned, the WCD may include a support structure configured to be worn by the patient such as, but not limited to, a garment (e.g., a vest). Included in the support structure of the WCD, a WCD monitor may include various components to facilitate the functionality of the WCD. A number of electrodes, monitoring electrodes and therapy electrodes, may be communicatively coupled with the WCD monitor. As the patient wears the WCD, the WCD may receive various data from the person such as, but not limited to, electrical signals in the form of electrocardiogram (ECG) signals, and the ECG signals (i.e., ECG data) may be stored by the WCD and/or grouped in accordance with various embodiments.

In this one non-limiting example scenario, the WMD may have multiple ECG vectors (i.e., multiple ECG signals from multiple electrodes). The multiple electrodes may be configured to facilitate monitoring of the heart rhythm of the patient. The WMD may analyze the ECG vectors to determine whether the heart rate (HR) of the person exceeds one or more arrhythmia thresholds such as, but not limited to, thresholds for VT or VF. If a threshold is exceeded, the WMD may open an episode in which the ECG data may be captured and stored for review by the clinician (e.g., the physician of the person). Additionally, the patient may experience several similar arrhythmias and/or noise events that may generate similar episodes. Accordingly, in some embodiments, the WMD may be configured to identify and group similar episodes to facilitate reduction of the burden on the clinician reviewing the episodes. For example, some of these embodiments may facilitate the clinician to review one of the episodes of a group of similar episodes and apply the assessment to the rest of the episodes in the group of similar episodes.

An example scenario, where numerous episodes may cause to be generated may include grouping of similar episodes may be due to one or more electrodes causing a noisier ECG signal (i.e., an ECG signal with noise above a threshold) or a lead-off (i.e., the ECG electrode not contacting the patient properly). In this scenario, the heart rhythm analysis may be halted or the heart rate may be falsely detected as a higher than VT rate threshold, which may cause the WMD to open an episode or issue one or more alarms. Noisier ECG signals may result in numerous episodes being detected, which may be a burden to the clinicians reviewing the episodes for diagnosis of potential heart issues.

In another example scenario, a patient wearing the WMD may be an active person (e.g., engaged in some form of physical exertion, exercising, playing sports, walking, lifting, running, etc.). The active patient may cause to generate numerous sinus tachycardia episodes due to the HR being accelerated during periods of physical exertion.

In yet another example scenario, a patient wearing the WMD may have an acute and/or heart related medical condition such as, but not limited to, chronic atrial fibrillation (AF) or atrial flutter (AFL). The patient wearing the WMD may cause to generate numerous similar episodes due to the HR associated with the AF or the AFL may be accelerated.

Continuing with the scenario of the WMD having multiple ECG vectors, at a given point in time, the multiple ECG vectors may cause to determine episodes due to a noisier ECG signal and/or a leadoff condition of one or more electrodes. The determined episodes may result in the WCD heart rhythm analysis being halted and/or or HR being falsely detected as higher than VT rate threshold that may cause the WCD to open an episode or an alarm. Ideally, determined episodes may be reviewed by the clinician and/or healthcare personnel. However, if noisier ECG signals cause numerous episodes to be determined for review by the clinician, the burden of the review process by the clinician may be increased. When numerous episodes are determined from noise or similar heart rhythms, one or more unique patterns for each patient may be determined. Accordingly, if the one or more unique patterns are identified early on, subsequent similar episodes may be screened out from review by the clinician to reduce the burden associated with the review process.

In another non-limiting example scenario, numerous episodes may be screened, sorted, filtered, and/or categorized at a receiving station, which may be located remote to the patient (e.g., a computing device at a medical facility, a clinic, emergency station, etc.). A clinician (e.g., medical professional, a physician, a caregiver, emergency personnel, etc.) may be able review a few episodes at the beginning of a wear time (i.e., the person may have put on the WMD and the WMD may have started to receive ECG data). The clinician may annotate one or more episodes as one of a listed events such as, but not limited to, noise events, AF, AFL, sinus tachycardia, other supraventricular tachycardia (SVT), VT, VF, and so forth as previously described.

In one example, a list of events/patterns may be provided to facilitate selection by the clinician to annotate one or more episodes. When the receiving station receives the numerous episodes, the receiving station may be configured to identify patterns associated with the received episodes and may determine a classification of the received episodes. If the first few episodes are labeled as the same or similar kind, a particular pattern associated with the same or similar kind of episodes may be identified (i.e., pattern matching approach). Having the same or similar kind of episodes identified may facilitate clinician to sort and/or view the episodes of interest rather than sifting through numerous episodes that may be not provide helpful information to the clinician.

Some examples of the pattern approach may include approaches based, at least in part, on measurements, which may have been performed by the WCD for each episode. For example, one or more of HR values, QRS widths, and other QRS measurements may be utilized. The pattern matching may depend on one or more parameters derived from ECG signals such as, but not limited to, HR, HR variability, QRS morphology, P-wave morphology, T-wave morphology, HR sudden onset, HR gradual onset, and so forth.

Some additional examples of the pattern approach may include approaches based, at least in part, on one or more noise measurements such as, but not limited to, measurement of noise amplitude and/or frequency including a combination of the previously described ECG based parameters. Some further additional examples of the pattern approach may include approaches based, at least in part, on one or more variety of physiological parameters such as respiration rate, blood pressure, oxygen saturation, and so forth, where the values of the one or more variety of physiological parameters may be utilized including a combination with one or more of the previously described ECG based and/or noise based parameters.

Additionally, the pattern matching approach may include the posture of the patient related measurements such as bending, lying on a side, walking, running, upper body movement, posture changes, and so forth of the patient. The pattern matching approach may include utilization of various position related technologies as will be described in more detail.

It should be appreciated that in some embodiments, the pattern matching approach includes approaches that may utilize a variety of measurements/information. Accordingly, the claimed subject matter is not limited in this respect.

In some examples, the pattern matching approach may include approaches based, at least in part, on determining a similarity utilizing features provided to group the episodes with similar patterns (e.g., machine learning and/or artificial intelligence based machine learning). For example, a machine learning may be applied to determine the similarities utilizing the ECG signals, acceleration signals, physiological signals, and so forth to group the episodes with similar patterns. The machine learning could include a linear regression, logistic regression, decision tree, support vector machine, naïve bayes, k-nearest neighbors, k-means, random forest, neural network, deep learning, other methods, and/or any combination thereof.

Utilizing the pattern matching approaches may facilitate association of future episodes with the same or similar patterns, which may be likely the same kind of episodes. As a result, clinical review may be streamlined to including skipping and/or reducing the priority for review by the clinician.

In some further examples of utilizing the pattern matching approaches, a pattern that may have been identified may be forwarded to a WMD to modify an algorithm. The modified algorithm may be configured to generate the episodes to reduce redundant episodes similar to the previously captured and stored episodes. Here again, clinical review may be streamlined to including skipping and/or reducing the priority for review by the clinician.

Having described some non-limiting example scenarios in connection with various embodiments, further detailed description of the various embodiments may be provided. The further detailed description may be provided with respect to some accompanying drawings.

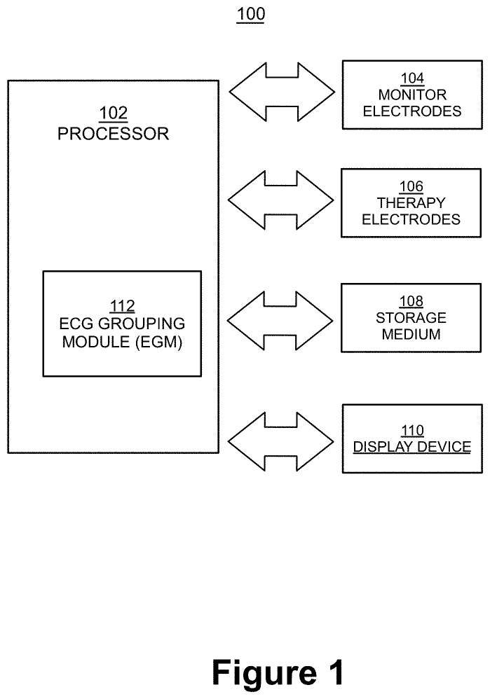

FIG. 1 illustrates a block diagram of a system for grouping ECG signals in accordance with various embodiments. In FIG. 1, a wearable medical device (WMD) system 100 may include a processor 102, one or more monitor electrodes (hereon, monitor electrodes 104), two therapy electrodes (hereon, therapy electrodes 106), and a storage medium 108. As shown, the monitor electrodes 104, the therapy electrodes 106, and the storage medium 108 may all be communicatively coupled to the processor 102 (i.e., under the control of the processor 102). Additionally, the WCD system 100 may include a display device 110. In FIG. 1, the processor 102 may include an ECG grouping module (EGM 112) in accordance with one or more embodiments.

In FIG. 1, the monitor electrodes 104 may be attachable to the skin of a person (shown in later figures) and may be configured to receive electrical signals from the person. The electrical signals may include ECG signals from the heart of the person. The ECG signals may be received by the processor 102, where the processor 102 may be configured to analyze the received ECG signals. The processor 102 may be configured to analyze the ECG signals to determine ECG signals that may be indicative of an arrhythmia of the heart of the person. The ECG signals determined to be indicative of an arrhythmia of the heart may be flagged as one or more episodes of arrhythmia. The processor 102 may store the one or more episodes in the storage medium 108.

Under the control of the processor 102, the EGM 112 may be configured to analyze the stored episodes to determine which of the episodes may meet at least one pattern matching criterion. The EGM 112 may associate into a group the episodes that may meet at least one pattern matching criterion. The EGM 112 may be configured to cause to display the group on the display device 110. As a result, a burden on the clinician in reviewing episode data may be reduced by enabling the clinician to review one of the episodes of a group of similar episodes and apply this assessment to the rest of the episodes in the group of similar episodes.

In FIG. 1, the various components of the WMD system 100 may be arranged in a variety of manners. Additionally, the WMD system 100 may include a wide variety of combinations of the components including omitting some of the components in accordance with various embodiments. For example, the therapy electrodes 106 may be components of a wearable cardioverter defibrillator (WCD). However, in some example embodiments, the WMD system 100 may not include the therapy electrodes and may only have the monitor electrodes 104. Accordingly, the claimed subject matter is not limited in these respects.

As shown in FIG. 1, the processor 102 having the EGM 112 may be communicatively coupled with the various components (e.g., monitor electrodes 104). However, in one example, the EGM 112 may be included in a processor that may be communicatively coupled with the various components wirelessly. For example, the EMG 112 may be included in a personal communication device such as, but not limited to, a smartphone. The smartphone may be communicatively coupled with the WMD system 100 wirelessly. The wireless communications may include a wide variety of wireless communication approaches such as, but not limited to, Bluetooth, Near Field Communication (NFC), WiFi, low power wireless communication (e.g., Zigbee), and so forth. Accordingly, the claimed subject matter is not limited in this respect.

In another example, the EGM 112 may be included in a processor that may be communicatively coupled from a remote location. For example, the EGM 112 may be included in a server type device. The server type device having the EGM 112 may be communicatively coupled with the WMD system 100 via a network. The network may be wired and/or wireless. In some examples, the network may be configured to provide connectivity for remote server type device and/or personal communication device. In some examples, the network may provide connectivity for the storage medium 108 and/or the display device 110, which may be communicatively coupled with the WMD system 100 wirelessly and/or wired. For example, the storage medium 108 may be a cloud based storage medium, the display device 110 may be located remote to the monitor electrodes 104 and the therapy electrodes 106 (i.e., remote from the WMD system 100), and so forth. Accordingly, the network may include a wide variety of communication mediums such as, but not limited to, the Internet, personal area networks (PAN), local area networks (LAN), wireless local area networks (WLAN), campus area networks (CAN), metropolitan area networks (MAN), wide area networks (WAN), storage-area networks (SAN), system-area networks, passive optical local area networks (POLAN), enterprise private networks (EPN), virtual private networks (VPN), and so forth. Accordingly, the claimed subject matter is not limited in this respect.

In FIG. 1, the processor 102 may be a wide variety of processors to facilitate at least some of the functionality described herein such as, but not limited to, machine learning capable processors. Some of examples of machine learning capable processors may include processors available from Intel Corporation of Santa Clara, Calif. (e.g., Nervana™ type processors), available from Nvidia Corporation of Santa Clara, Calif. (e.g., Volta™ type processors), available from Apple Company of Cupertino, Calif. (e.g., A11 Bionic™ type processors), available from Huawei Technologies Company of Shenzen, Guangdong, China (e.g., Kirin™ type processors), available from Advanced Micro Devices, Inc. of Sunnyvale, Calif. (e.g., Radeon Instinct™ type processors), available from Samsung of Seoul, South Korea (e.g., Exynos™ type processors), and so forth. Accordingly, the claimed subject matter is not limited in this respect.

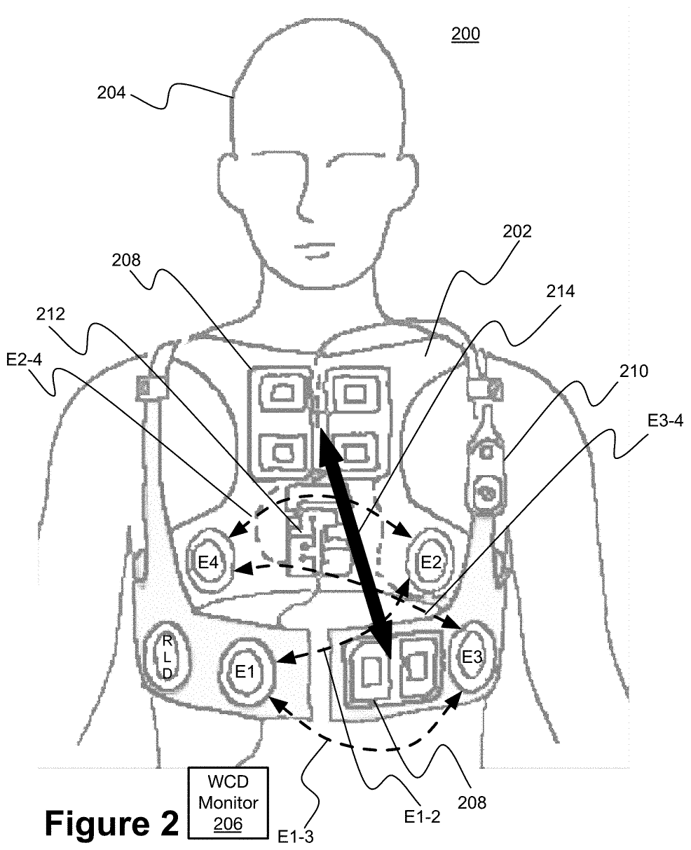

FIG. 2 illustrates an example of a medical device that may be utilized, in accordance with at least one or more example embodiments. In FIG. 2, a medical device may be a wearable medical device (WMD), which may be configured to facilitate monitoring and treatment of a heart of a person (not shown) such as, but not limited to, a wearable cardioverter defibrillator (WCD) 200. The WCD 200 may be included in or attached to a support structure 202, which may be configured to be worn by a person 204.

In FIG. 2, the WCD 200 may include various electronic components to facilitate the functionality of the WCD 200 as a heart monitoring and defibrillator device including grouping of electrocardiogram (ECG) signals of the heart of the person 204 wearing the WCD 200 as described herein. The various electronic components may be included and illustrated as a WCD module (hereon a WCD monitor 206). The WCD monitor 206 may be communicatively coupled with the EGM 112 (as shown in FIG. 1) and may include components to facilitate to the various functionalities as described herein in accordance with various embodiments. The WCD 200 may include two therapy electrodes configured to defibrillate the heart of the person, defibrillator electrodes 208, and a number of monitoring electrodes E1, E2, E3, and E4 disposed as shown and configured to detect and measure the electrical activity of the heart (e.g., ECG). Additionally, the WCD 200 may include a Right Leg Drive (RLD) electrode, where the RLD electrode may be configured to manage electrical signal noise such as, but not limited to, common mode noise off the WCD 200.

As shown in FIG. 2, the support structure 202 may include additional components that may be configured to determine various health related information other than measuring electrical activity of the heart. For example, the WMD 200 may include an additional component such as, but not limited to, a component configured to facilitate measurement of oxygen levels in the blood of the person (e.g., pulse oximetry/SpO2 device 210).

In FIG. 2, the WCD 200 may have one or more sensors 212 that may be included in the support structure 202. The one or more sensors 212 may be communicatively coupled with the WCD monitor 206 to facilitate at least some of the functionalities as described herein. For example, the one or more sensors 212 may include accelerometer type sensors that may be configured to detect motion and/or position of the WCD 200, which in turn may facilitate determination of motion and/or position (e.g., posture) of the person 204. The determined motion and/or posture of the person may be provided to the EGM 112 to be processed.

In one example, the number of monitoring electrodes E1, E2, E3, and E4 shown in FIG. 2 may be single-ended monitoring electrodes. In this example, the single-ended monitoring electrodes E1, E2, E3, and E4 may include differential vectors/channels Ch1 E1-2 (vector/channel between monitoring electrodes E1 and E2), Ch2 E1-3 (vector between monitoring electrodes E1 and E3), Ch5 E2-4 (vector between monitoring electrodes E2 and E4), and Ch6 E3-4 (vector between monitoring electrodes E3 and E4). The differential vectors/channels Ch1 E1-2, Ch2 E1-3, Ch5 E2-4, and Ch6 E3-4 may be derived from the single-ended vectors of the single-ended monitoring electrodes E1, E2, E3, and E4.

In some examples, the defibrillator electrodes 208 may be disposed in the support structure 202 to be proximate to a left front of the person 202 and a back of the person to facilitate a defibrillation shock vector 214 as shown in FIG. 2.

The person 204 may be ambulatory (i.e., capable of moving and not bedridden). Movement by the person 204 may affect the ECG signals detected by the Ch1 E1-2, Ch2 E1-3, Ch5 E2-4, and Ch6 E3-4 (from electrodes E1, E2, E3, and E4). For example, movement by the person 204 may cause noise in the ECG signals generated at an electrode-skin interface at one or more of the monitoring electrodes E1, E2, E3, and E4. The noise in the ECG signals may negatively affect interpretation of the ECG signals (e.g., erroneous interpretation of potential issues with the heart).

Some example effects may include, but not limited to, false detection of a leadoff condition at one or more of the monitoring electrodes E1, E2, E3, and E4 and/or at one or more of the defibrillator electrodes 208 (i.e., contact with the skin of the person 204 may be below a predetermined threshold, where the predetermined threshold may be prescribed by the WCD 200 manufacturer to ensure proper functionality of the WCD 200). For example, a leadoff condition may cause the WCD 200 to halt rhythm analysis or may cause the WCD 200 to open a heart related episode or an alarm due to the false determination of the heart rate being detected as higher than a VT rate threshold. It should be appreciated that most heart related episodes may be reviewed by a clinician or healthcare personnel, and accordingly, if the ECG signals include noise, which may cause too many episodes for clinical review, the clinician or healthcare personnel may be burdened with reviewing of the one or more false indications.

The support structure 202 may be implemented as some examples described for support structures of US Pat. App. No. US2017/0056682, which is incorporated herein by reference for all purposes. As may be appreciated, the person skilled in the relevant art will recognize that additional components of the WMD 200 may be included in a housing of the support structure 202 instead of being attached externally to the support structure as may be described in the US2017/0056682 document

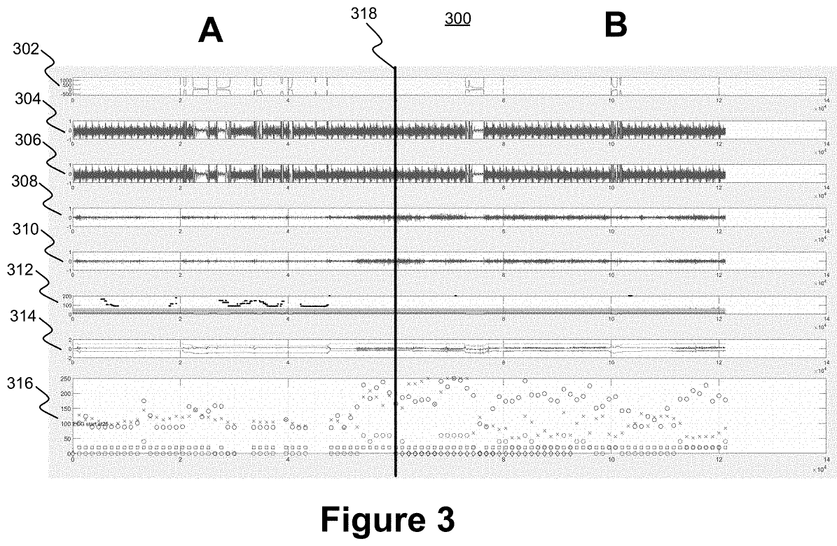

FIG. 3 illustrates an example of an output of various signals from a WMD in accordance with various embodiments. In FIG. 3, a sample output 300 may include numerous outputs. For example, the sample output 300 may include a first output 302, which may include information such as, but not limited to, Unfiltered ECG+Common including date and time. The sample output 300 may include a number of outputs from various channels, which may be shown as a second output 304 having information such as, but not limited to, Ch1 filteredECG, a third output 306 having information such as, but not limited to, Ch2 filteredECG, a fourth output 308 having information such as, but not limited to, Ch5 filteredECG, and a fifth output 310 having information such as, but not limited to, Ch6 filteredECG.

In FIG. 3, the sample output 300 may include a sixth output 312 having information such as, but not limited to, Gatekeeper HR LeadOff(b) DefibLeadOff(r). A seventh output 314 may have information such as, but not limited to, Accelerometer Signal. Additionally, the sample output 300 may include an eighth output 316 having information such as, but not limited to, RRD results HR(ko), Width(rx), Anynoise (ks) Rhythm(ro) SVT=20, VT=40, VF=60, MFK(kd) Formed.

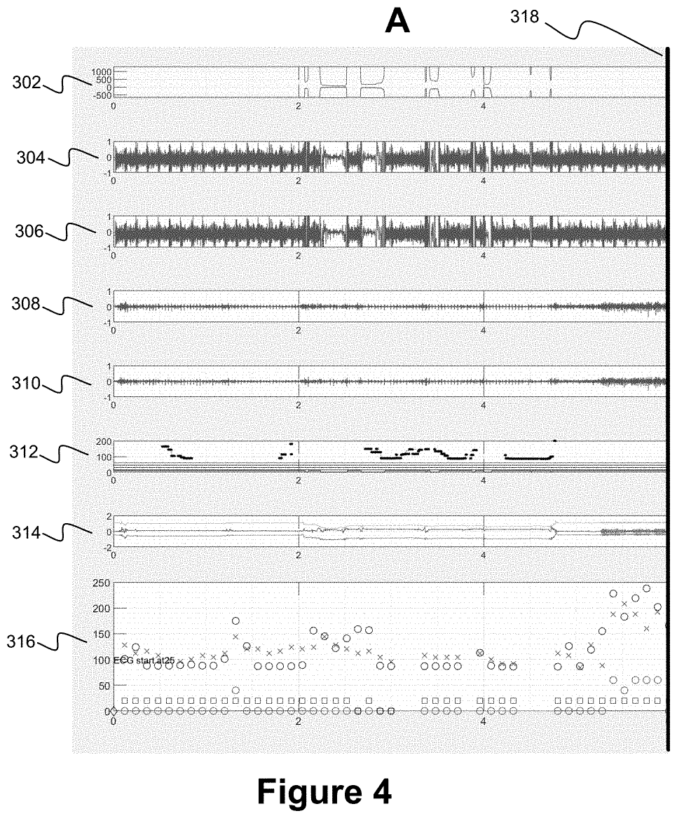

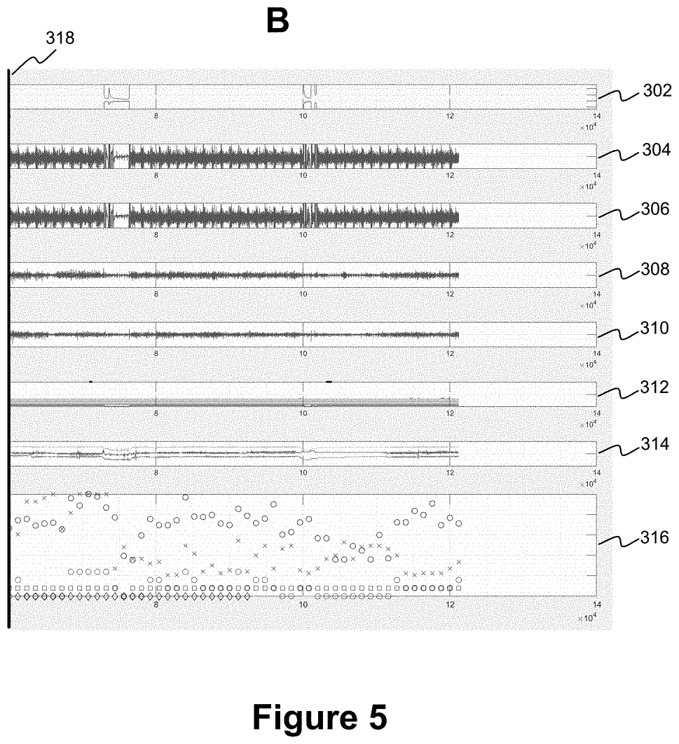

In FIG. 3, the sample output 300 includes a dividing line 318. The dividing line 318 indicates the sample output 300 being divided into two separate parts, A and B. The two separate parts A and B are shown in FIGS. 4 and 5 respectively.

FIG. 4 shows part A of the sample output 300 (shown in FIG. 3) in further detail. Accordingly, part A includes the number of outputs 302, 304, 306, 308, 310, 312, 314, and 316 described in FIG. 3.

FIG. 5 shows part B of the sample output 300 (shown in FIG. 3) in further detail. Accordingly, part B includes the number of outputs 302, 304, 306, 308, 310, 312, 314, and 316 described in FIG. 3.

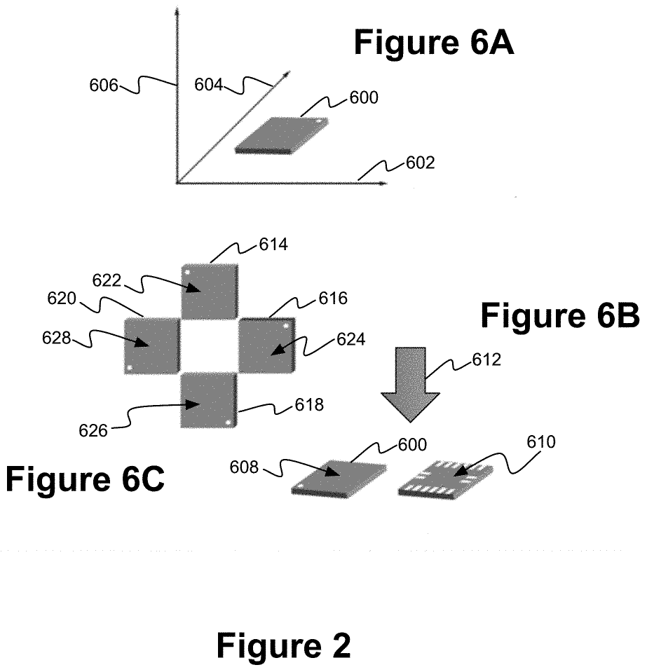

FIGS. 6A-6C illustrate an example of an accelerometer configuration, which may be utilized with various embodiments. In FIG. 6A, an accelerometer 600 may have three axes of acceleration sensitivity A_{X} 602, A_{Y} 604, and A_{Z} 606 (i.e., acceleration along the one or more of the axes of acceleration sensitivity A_{X}, A_{Y}, and A_{Z} may cause the corresponding output to increase).

FIG. 6B illustrates orientation of the accelerometer 600 with respect to gravity. In FIG. 6B, the accelerometer 600 is shown having a top side 608 and a bottom side 610. As shown, the direction of gravity 612 may be towards the top side 608 of the accelerometer 600. In this example configuration, the output response for 1 g of gravity may be as follows:
on the top side 608 of the accelerometer 600, Xₒᵤₜ=0 g; Yₒᵤₜ=0 g; Zₒᵤₜ=1 g
on the bottom side 610 of the accelerometer 600, Xₒᵤₜ=0 g; Yₒᵤₜ=0 g; Zₒᵤₜₜ=−1 g.

FIG. 6C illustrates an example of a configuration of a number of accelerometers. In FIG. 6c, four accelerometers 614, 616, 618, and 620 may be configured in a checkered pattern (i.e., four substantially squarish shaped accelerometers 614, 616, 618, and 620 arranged corner to corner). The four accelerometers 614, 616, 618, and 620 may be shown with a top side 622, 624, 626, and 628 of each of the accelerometers 614, 616, 618, and 620 facing out of the page.

In the configuration shown in FIG. 6C, the output response for 1 g of gravity may be as follows:
at the first accelerometer 614, Xₒᵤₜ=−1 g; Yₒᵤₜ=0 g; Zₒᵤₜ=0 g
at the second accelerometer 616, Xₒᵤₜ=0 g; Yₒᵤₜ=−1 g; Zₒᵤₜ=0 g
at the third accelerometer 618, Xₒᵤₜ=1 g; Yₒᵤₜ=0 g; Zₒᵤₜ=0 g
at the first accelerometer 620, Xₒᵤₜ=0 g; Yₒᵤₜ=1 g; Zₒᵤₜ=0 g.

Accordingly, the output response versus orientation of the accelerometer to gravity may be described with respect to the configuration of the accelerometers shown in FIGS. 6A-6C and may be utilized with the various embodiments.

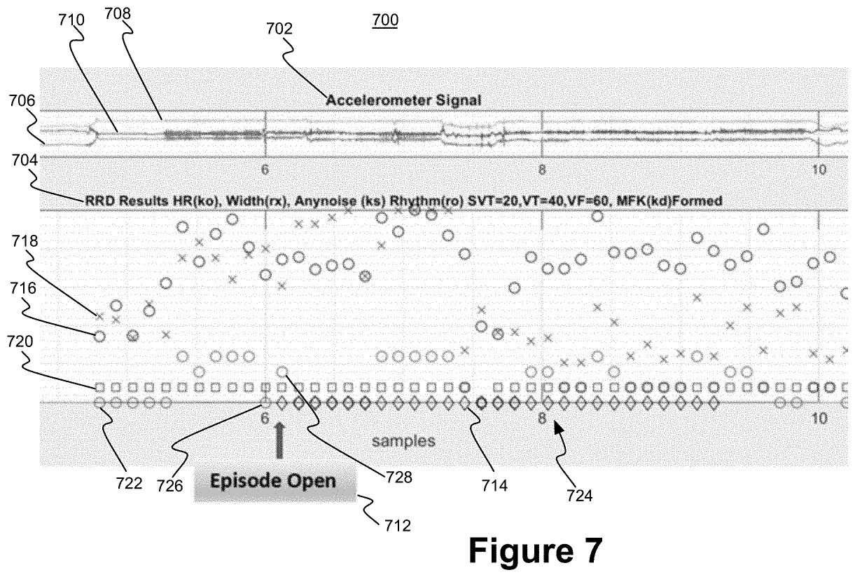

Turning now to FIG. 7, FIG. 7 is a portion of a sample output showing some further detail in accordance with various embodiments. The portion of the sample output 700 may be a portion of the sample output 300 (shown in FIG. 3). In FIG. 7, the portion of the sample output 700 may be a result of signals from an accelerometer 702 and heart related signals 704. In one example, the WCD may be configured to be dispose as a 3-axis accelerometer that may be positioned on the back of the person 204 shown in FIG. 2 (e.g., between the shoulder blades of the person 204). In this example configuration, the Y axis may represent the up and down acceleration of the person 204 (e.g., when the person 204 stands or sits straight up). The output of this type of motion may be indicated by a first accelerometer signal 706 (if in color, red). The Z axis may represent a front and back orientation of the person 204 (e.g., when the person 204 may be lying on the back (supine) or on the chest (prone) or bending forward). The output of this type of motion may be indicated by a second accelerometer signal 708 (if in color, yellow). The X axis may represent a right and left orientation of the person 204 (e.g., when the person may be lying on the right side or on the left side). The output of this type of motion may be indicated by a third accelerometer signal 710 (if in color, blue).

Continuing to refer to FIG. 7, the portion of the sample output 700 may cause an episode to be opened. For example, the posture of the person 204 may have changed to a prone posture, which may cause the measured HR to be elevated due to the motion of the posture change, which may be related to noise in the ECG signal. Referring to the signals from an accelerometer 702 having the first accelerometer signal 706, second accelerometer signal 708, and the third accelerometer signal 710, an episode 712 may be opened, which may correspond to the change in posture of the person 204. In the sample output 700, the signals from the accelerometer 702 may have a range of +/−2 g or +/−4 g. As shown in FIG. 7, the sample output 700 show the heart related signals 704 having numerical values associated with the episode 712, where each time point may represent one result of one segment analysis. As shown in FIG. 7, the sample output 700 may display data that may show a time period, which may start prior to the episode 712 and may extend past the end of the episode 712. In FIG. 7, a black diamond 714 may represent a segment that may be part of the episode 712, a black circle 716 may represent a heart rate value, a red X 718 may represent a QRS width, a black square 720 may represent an indication of the presence or absence of noise detection, and a red circle 722 may represent different rhythm types. In the example shown in FIG. 1, if a red circle 722 is at a value of 20, a supraventricular tachycardia (SVT) may be indicated, if a red circle 722 is at a value of 40, a VT may be indicated, and if a red circle 722 is at a value of 60, a VF may be indicated. In the sample output 700, a horizontal axis scale 724 may be 500 samples per second of ECG signals.

As shown in FIG. 7, at approximately six data points or segments prior to the episode 712, the signals from the accelerometer 702 having the first accelerometer signal 706, second accelerometer signal 708, and the third accelerometer signal 710 may show that the person 204 was moving. Correspondingly, the black circles 716 (i.e., measured HR) and the red Xs 718 (i.e., measured QRS widths) may be shown to increase. In the example shown in FIG. 7, after approximately, 6 data point or segments, an arrhythmia analysis algorithm of a WCD may have processed the various received data (e.g., ECG signals and/or QRS widths) and detected VT, which may be shown by the red circles 722 as being increased from a value of zero 726 to a value of 40 728. Accordingly, the arrhythmia analysis algorithm may have opened the episode 712. As previously described, the increases in measured HR and measured QRS width may be the result of the movements of the person 204 moving to the prone position. Some example embodiments for detecting when the person 204 is in a prone or supine position (e.g., posture detection) may be described further below.

In the example shown in FIG. 7, the movement of the person 204 moving to the prone position may have added noise to the ECG signals, which may have affected the HR and QRS width being larger than actual values. The HR and QRS widths being larger values may result in VT indications causing the episode 712 to be opened.

In some examples, the signals from the accelerometer 702 having the first accelerometer signal 706, second accelerometer signal 708, and the third accelerometer signal 710 and noise may be utilized by the WCD or a WCD system to analyze subsequent episodes to detect similar scenarios and group them together to facilitate a clinician (e.g., healthcare personnel) to review the group of episodes easily. For example, in example embodiments, episodes may be uploaded to a server for review by clinicians (e.g., healthcare personnel, physician, etc. of the person 204). The server may be configured to analyze the uploaded episodes to identify and group episodes matching a pattern, thereby reducing a burden on the clinician.

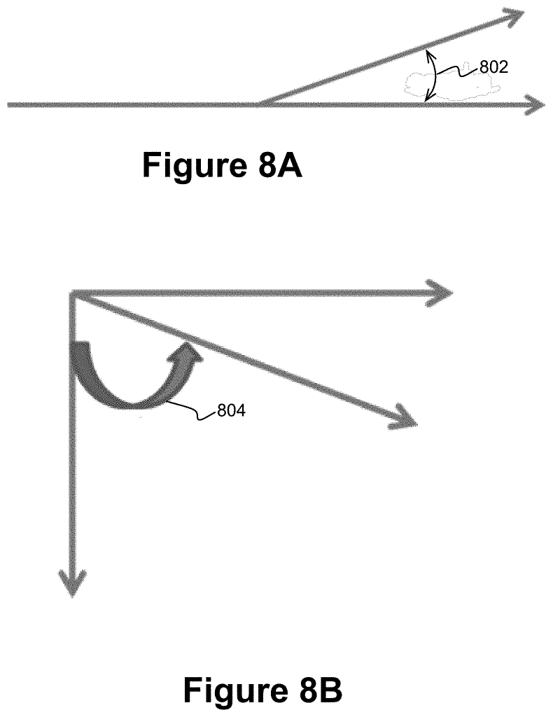

FIGS. 8A and 8B illustrate some examples of determining a posture of a person in accordance with various embodiments. In FIG. 8A, a posture of a person may be determined to be in a lying position based, at least in part, on an angle 802. For example, if the angle 802 is less than 30 degrees, it may be determined that the person is in a lying position.

In FIG. 8B, an angle 804 may be measured from an upright position. For example, if the angle 804 is substantially equal to 0, it may be determined that the person is standing in an upright position. If the angel 804 is substantially 90 degrees, it may be determined that the person is in a supine position. Accordingly, in some examples, it may be determined that the person is in a lying position if −1*cos(60)=−0.5<Y<1*cos(60)=0.5. In some examples, it may be determined that the person is standing if Y<−1*cos(20)=−0.9397. In the previous examples, it may be assumed that the variable Y=−1.

Determination of changes in posture may be utilized to determine motion of the person. For example, a slow, normal posture change (e.g., the person getting into bed and lying down) may be determined when an axis may show a significant difference over a period of time. For example, if the normal posture changes over 5 seconds is greater than a threshold, for example 0.5G, the posture change is determined.

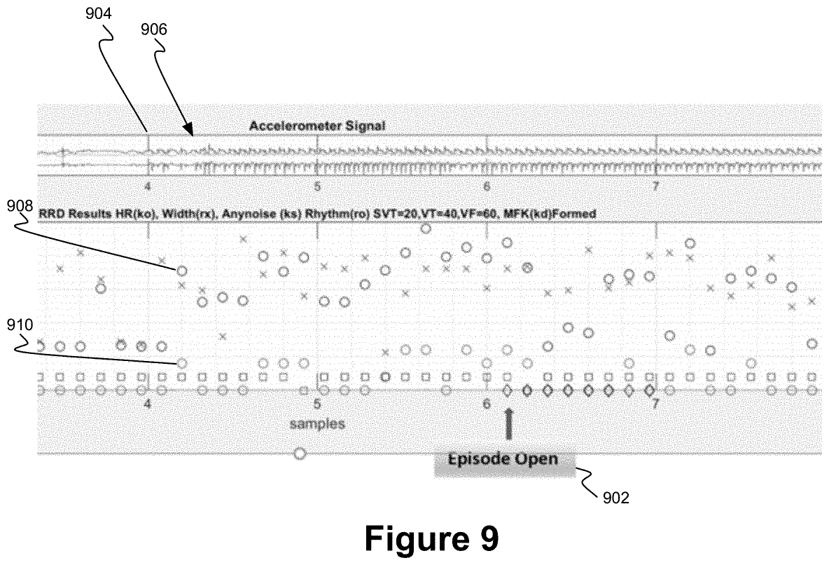

FIG. 9 illustrates various signals, which may be related to an activity of a person in accordance with various embodiments. In FIG. 9, a person may be performing a regular activity such as, but not limited to, walking in a regular manner. As the person walks, the HR of the person may be accelerated, which may cause an episode 902 to be opened. The episode 902 may be opened due to noise in the ECG signals, which may have been caused by the normal activity of walking by the person.

As shown in FIG. 9, a little past a 40K sample mark 904 the accelerometer signals 906 may indicate accelerations, which may be caused by movements related to the person walking. As can be seen, a measured HR may increase (i.e., elevated HR 908) due to the activity off the person (i.e., walking). Some increase in HR may be considered normal, but the elevated HR 908 may be due to noise, which may be caused by movement of one or more ECG electrodes as the person engages in the activity (i.e., walking). The elevated HR 908 may cause an elevated QRS 910, which may result in an indication of VT (e.g., the red circles increasing to a value of 40). In this example, the indication of VT may be erroneous. Accordingly, in the various embodiments, the pattern of accelerometer signals 906 and the noise (e.g., the elevated HR 908 and the elevated QRS 910) may be utilized by the WCD to analyze subsequent episodes to detect similar scenarios and group them together, thereby facilitating a clinician to review more easily or quickly these type of episodes.

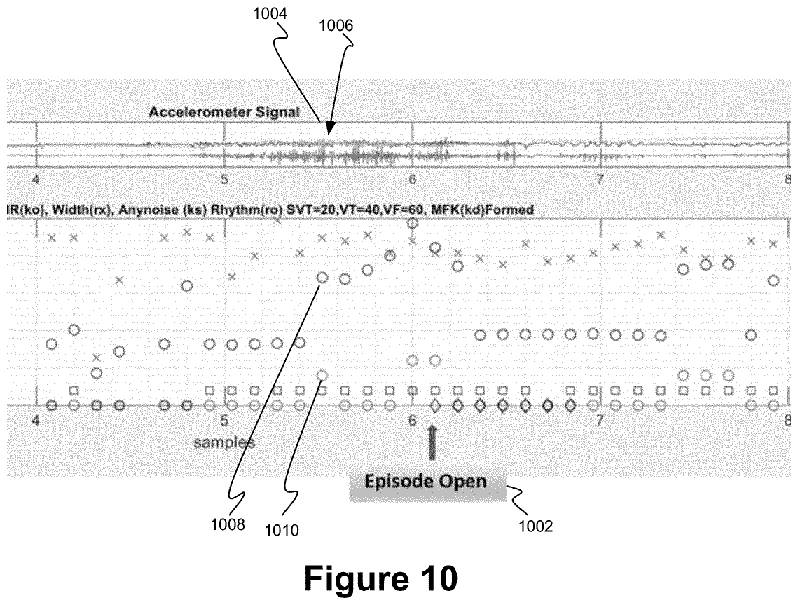

FIG. 10 illustrates signals received from a person, who may be in a moving vehicle in accordance with various embodiments. In FIG. 10, a person may be in a vehicle such as, but not limited to, an automobile. The person may be driving the automobile. As shown in FIG. 10, an episode 1002 may be opened due to detection and determination of some form of health condition (e.g., VT). For example, at approximately 55K sample mark 1004, accelerometer signals 1006 may indicate relatively high accelerations. At approximately the 55K sample mark 1004, the received signals may indicate an elevated HR 1008 and an elevated QRS 1010, which may be determined to indicate VT condition.

In FIG. 10, the episode 1002 (e.g., determination of indication of VT) may be erroneous due to noise, which may be caused by movements of the vehicle being imparted onto the person (i.e., forces imparted on the person causing the elevated HR 1008 and the elevated QRS 1010). The elevated HR 1008 and the elevated QRS 1010 may have caused the episode 1002 to be opened approximately 6 data points subsequent. The pattern of the accelerometer signals 1006 and noise (i.e., elevated HR 1008 and the elevated QRS 1010) may be utilized by the WCD to analyze subsequent episodes to detect similar scenarios and group them together facilitating the clinician to review these types of episodes more easily or quickly.

Prior to describing further embodiments and/or examples, it should be appreciated that even though some description included examples of WCDs, the claimed subject matter may be applicable to examples of a wide variety of cardiac monitoring devices. Accordingly, the claimed subject matter is not limited in these respect.

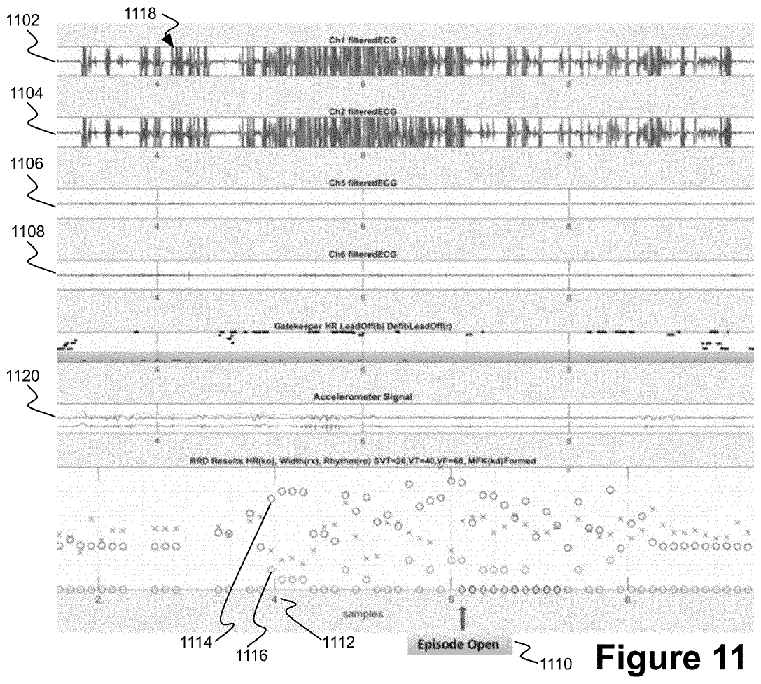

FIG. 11 illustrates signals related to potential issues with one or more ECG electrodes. In the example of FIG. 11, an ECG electrode of a multi-channel system may have contact issues with a skin of a person (i.e., a lead-off condition). In this example, a WCD may include four channels (a first channel 1102, a second channel 1104, a third channel 1106, and fourth channel 1108). As shown in FIG. 11, the lead-off condition of one ECG electrode may affect at least two of the four channels (i.e., the first channel 1102 and the second channel 1104). In some examples, even though the WCD may continue to operate having the lead-off condition, the noise caused by the lead-off condition may undesirably cause noise triggered episodes (e.g., VT) similar to the previously described episodes.

In FIG. 11, an episode 1110 may be opened due to detection and determination of some form of health condition (e.g., VT). For example, at approximately 40K sample mark 1004, an elevated HR 1114 and an elevated QRS 1116 may be detected. Additionally, noise 1118 on the first channel 1102 and the second channel 1104 may be shown to increase. However, in this example, the elevated HR 1114 and the elevated QRS 1116 may not correlate with acceleration signals 1120. The pattern shown in FIG. 11 may be indicative of a lead-off condition rather than noise caused by movement of the person. For this example, an algorithm may recognize as a lead-off pattern when multiple ECG channels are saturated with noise 1118, while the accelerometer signals 1120 may not indicate movement of the person. In some other examples, the lead-off condition for each channel 1102, 1104, 1106, and 1108 may be included in the pattern matching algorithm. This pattern of accelerometer signals 1120 and noise 1118 may be utilized by the WCD to analyze subsequent episodes to detect similar scenarios and group them together facilitating a clinician to review these types of episodes more easily or quickly.

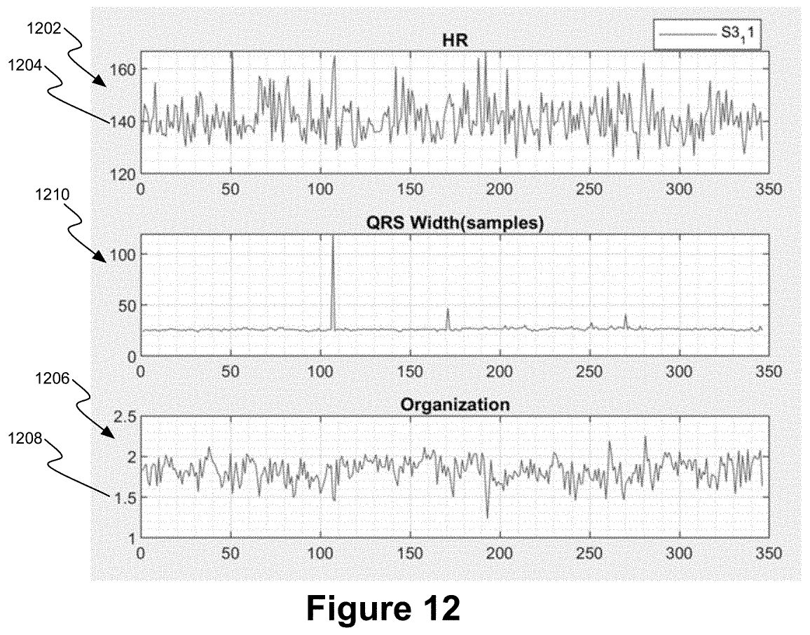

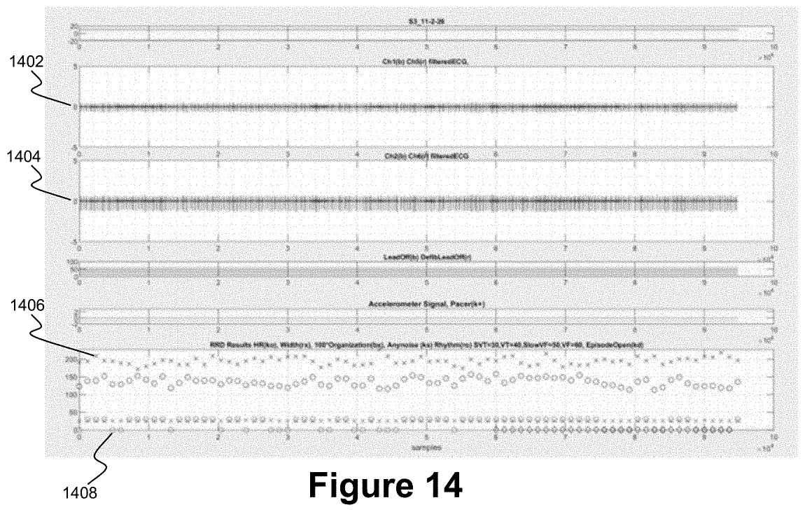

FIG. 12 illustrates signals related to HR and QRS signals in accordance with various embodiments. In FIG. 12, HR signals 1202 may indicate somewhat varying around the mean value of 140 bpm 1204. Additionally, as shown, QRS organization 1206 may indicate varying around a mean value of 1.5 1208, while QRS width 1210 may be relatively consistent. In some examples, QRS organization may be determined as disclosed in U.S. Pat. No. 10,016,614 filed Jan. 31, 2017, entitled “Wearable Cardioverter Defibrillator (WCD) System Making Shock/No Shock Determinations by Aggregating Aspects of Multiple Patient Parameters”, which is incorporated herein by references for all purposes. The parameters shown in FIG. 12 may be derived from ECG signals as may be illustrated in FIG. 14 below.

The signals related to HR 1202, QRS organization 1206, and QRS width 1210 may be indicative of AF. In some examples, the pattern of signals shown in FIG. 12 may be utilized by a WCD and/or a WCD system to analyze subsequent episodes to facilitate detection of similar scenarios and group them together allowing a clinician to review these types of episodes more easily or quickly. For example, if a first few episodes of a group of episodes matching the pattern of signals (e.g., HR 1202, QRS organization 1206, and QRS width 1210) are confirmed, an indication of AF may be determined. Accordingly, subsequent episodes having confirmed similar pattern of signals (e.g., HR 1202, QRS organization 1206, and QRS width 1210) around similar mean values indicating AF rhythms may be utilized by a reviewing clinician to consider the remaining episodes in the group as the same AF rhythms and can quickly review and/or even skip reviewing the remaining episodes in the group.

It should be appreciated that in this example, all three parameters (HR 1202, QRS organization 1206, and QRS width 1210) may be utilized in the pattern matching to group episodes. However, in some examples only the QRS width 1210 may be utilized for the pattern matching purposes in accordance with various embodiments.

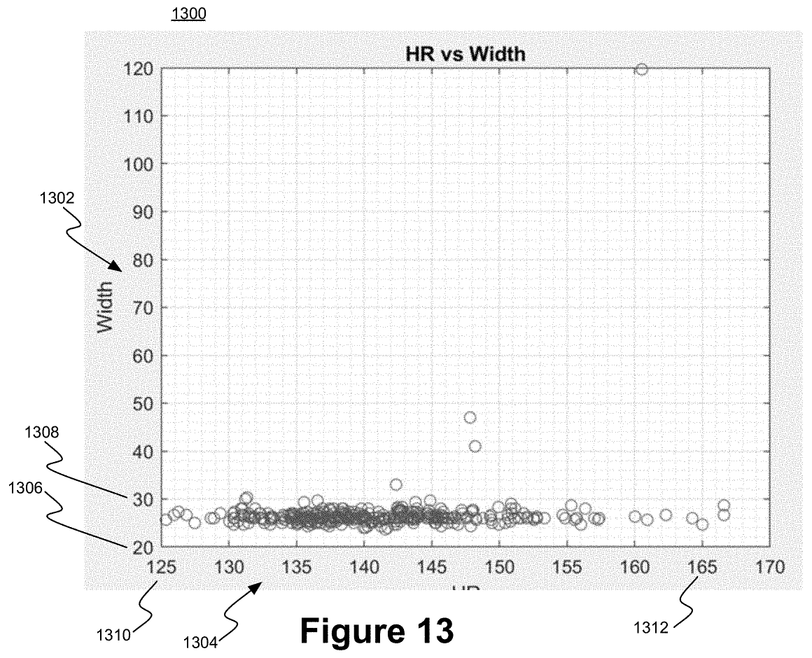

FIG. 13 illustrates QRS widths signals in more detail. As mentioned above, in the example shown in FIG. 12, the QRS width 1210 may be relatively consistent. The consistency may be shown in FIG. 13. In FIG. 13, a graph 1300 may include a vertical axis showing QRS width (QRS width 1302) and a horizontal axis showing HR (HR 1304). As mentioned above with respect to FIG. 12, the graph 1300 may be derived from ECG signals illustrated in FIG. 14.

As shown in FIG. 13, the exception of only a few data points, the QRS width 1302 may be consistently between 20 ms 1304 and 30 ms 1308 for HR 1304 ranging from 125 bps 1310 to 166 bps 1312. In some examples, the metric shown in the graph 1300 may be utilized in pattern matching of episodes to group similar episodes together.

FIG. 14 illustrates ECG signals of an episode that may be utilized to derive the charts of FIGS. 12 and 13. FIG. 14 may be similar to previously described FIG. 11. In FIG. 14, multi-channel ECG signals may include a first channel 1402 and a second channel 1404. Additionally, FIG. 14 may include a consistency data 1406 and a modification of rhythm parameters 1408. In the example shown in FIG. 14 a modification of rhythm parameters 1408 at 30 may indicate SVT, a modification of rhythm parameters 1408 at 40 may indicate VT, a modification of rhythm parameters 1408 50 may indicate a slow VT, and a modification of rhythm parameters 1408 at 60 may indicate VF. In some examples, the consistency data 1406 may be determined as described in US patent application U.S. Ser. No. 15/421,165, filed Jan. 31, 2017 (now issued as U.S. Pat. No. 10,016,614) titled Wearable cardioverter defibrillator (WCD) system making shock/no shock determinations by aggregating aspects of multiple patient parameters, which is incorporated by reference in its entirety for all purposes.

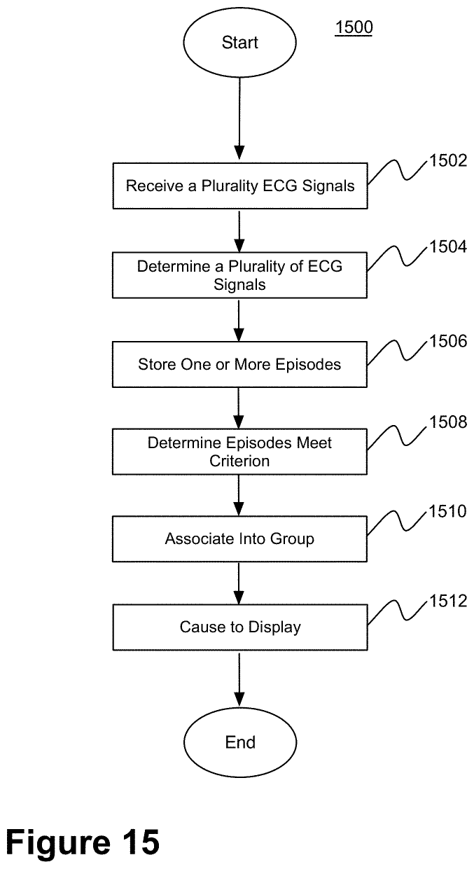

FIG. 15 illustrates an operational flow for grouping of electrocardiogram (ECG) signals of a heart of a person (e.g., patient) wearing a wearable medical device (WMD) in accordance with various embodiments as described herein. In some portions of the description, illustrative implementations of the method are described with reference to the elements depicted in FIGS. 1-14. However, the described embodiments are not limited to these depictions.

Additionally, FIG. 15 employs block diagrams to illustrate the example methods detailed therein. These block diagrams may set out various functional block or actions that may be described as processing steps, functional operations, events and/or acts, etc., and may be performed by hardware, software, and/or firmware. Numerous alternatives to the functional blocks detailed may be practiced in various implementations. For example, intervening actions not shown in the figures and/or additional actions not shown in the figures may be employed and/or some of the actions shown in one figure may be operated using techniques discussed with respect to another figure. Additionally, in some examples, the actions shown in these figures may be operated using parallel processing techniques. The above described, and other not described, rearrangements, substitutions, changes, modifications, etc., may be made without departing from the scope of the claimed subject matter.

In some examples, operational flow 1500 may be employed as part of WMD system having communication capabilities including a personal communication device, a server, a wireless communication network, and so forth as described herein. Beginning at block 1502 (“Receive a Plurality ECG Signals”), an electrocardiogram (ECG) grouping module (EGM) may receive a plurality of ECG signals from a person wearing a wearable medical device (WMD) via a plurality of electrodes adapted to be in contact with the person.

Continuing from block 1502 to block 1504 (“Determine a Plurality of ECG Signals”), the EGM may determine a plurality of ECG signals indicative of an arrhythmia of a heart of the person from the plurality of received ECG signals.

Continuing from block 1504 to block 1506 (“Store One or More Episodes”), the EGM may store one or more episodes of the determined plurality of ECG signals indicative of an arrhythmia.

Continuing from block 1506 to block 1508 (“Determine Episodes Meet Criterion”), the EGM may determine which of the stored one or more episodes meet at least one pattern matching criterion.

Continuing from block 1508 to block 1510 (“Associate Into Group”), the EGM may associate into a group the determined one or more episodes that meet at least one pattern matching criterion.

Continuing from block 1510 to block 1512 (“Cause to Display”), the EGM may cause to display the group. The displayed group may be utilized by a reviewing clinician as described herein in various embodiments.

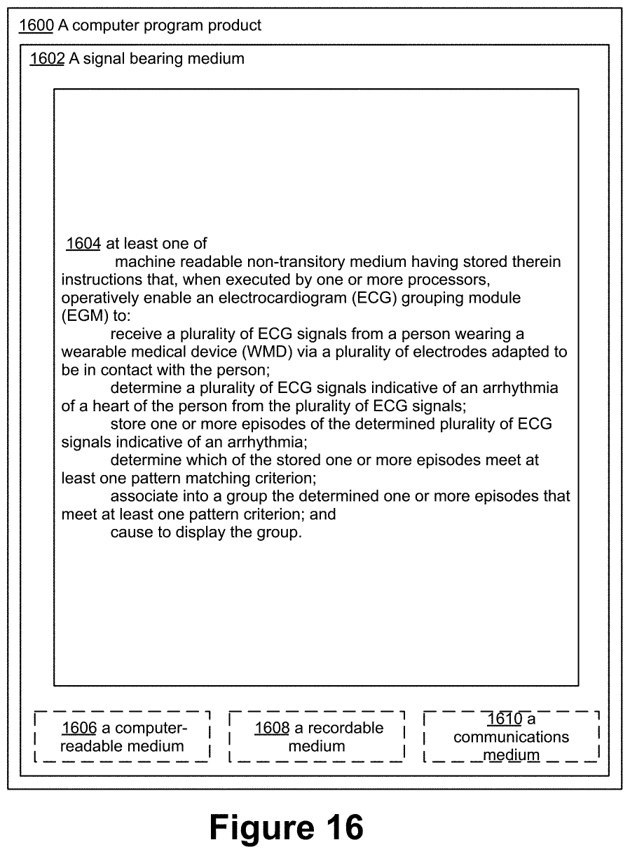

In general, the operational flow described with respect to FIG. 15 and elsewhere herein may be implemented as a computer program product, executable on any suitable computing system, or the like. For example, a computer program product for facilitating grouping of ECG signals of a heart of a person (e.g., patient) wearing a WMD may be provided. Example computer program products may be described with respect to FIG. 16 and elsewhere herein.

FIG. 16 illustrates an example computer program product 1600, arranged in accordance with at least some embodiments described herein. Computer program product 1600 may include machine readable non-transitory medium having stored therein instructions that, when executed, cause the machine to facilitate grouping of ECG signals of a heart of a person (e.g., patient) wearing a WMD according to the processes and methods discussed herein. Computer program product 1600 may include a signal bearing medium 1602. Signal bearing medium 1602 may include one or more machine-readable instructions 1604 which, when executed by one or more processors, may operatively enable a computing device to provide the functionality described herein. In various examples, the devices discussed herein may use some or all of the machine-readable instructions.

In some examples, the machine readable instructions 1604 may include an electrocardiogram (ECG) grouping module (EGM) configured to facilitate grouping of ECG signals of a heart of a person (e.g., patient) wearing a WMD. In some examples, the machine readable medium 1604 may facilitate the EGM to receive a plurality of ECG signals from a person wearing the WMD via a plurality of electrodes adapted to be in contact with the person, determine a plurality of ECG signals indicative of an arrhythmia of a heart of the person from the plurality of received ECG signals, store one or more episodes of the determined plurality of ECG signals indicative of an arrhythmia, determine which of the stored one or more episodes meet at least one pattern matching criterion, associate into a group the determined one or more episodes that meet at least one pattern matching criterion, and cause to display the group.

In some implementations, signal bearing medium 1602 may encompass a computer-readable medium 1606, such as, but not limited to, a hard disk drive, a Compact Disc (CD), a Digital Versatile Disk (DVD), a Universal Serial Bus (USB) drive, a digital tape, memory, etc. In some implementations, the signal bearing medium 1602 may encompass a recordable medium 1608, such as, but not limited to, memory, read/write (R/W) CDs, R/W DVDs, etc. In some implementations, the signal bearing medium 1602 may encompass a communications medium 1610, such as, but not limited to, a digital and/or an analog communication medium (e.g., a fiber optic cable, a waveguide, a wired communication link, a wireless communication link, etc.). In some examples, the signal bearing medium 1602 may encompass a machine readable non-transitory medium.

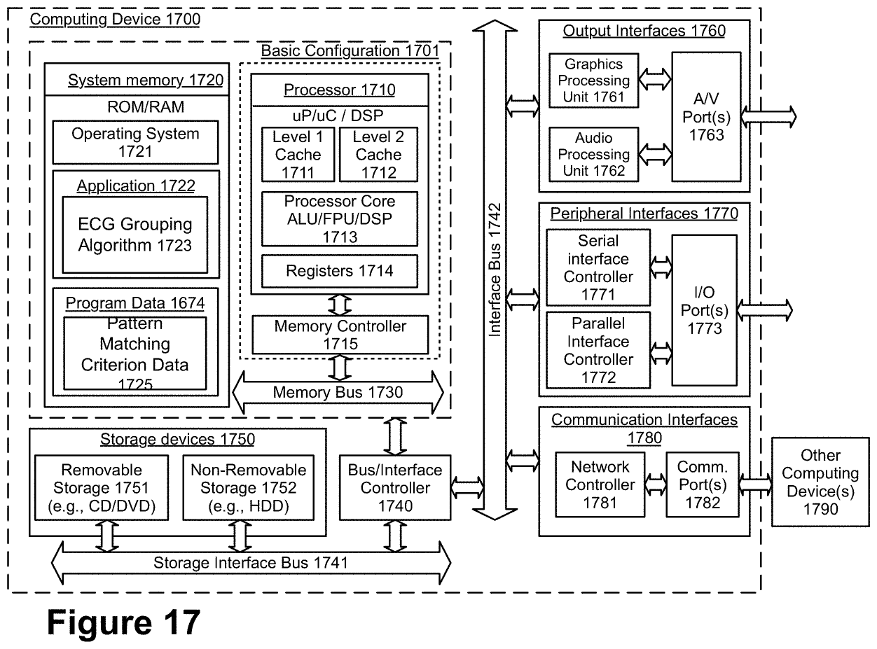

In general, the methods described with respect to FIG. 16 and elsewhere herein may be implemented in any suitable computing system. Example systems may be described with respect to FIG. 17 and elsewhere herein. In general, the system may be configured to facilitate a EGM in accordance with various embodiments.

FIG. 17 is a block diagram illustrating an example computing device 1700, such as might be embodied by a person skilled in the art, which is arranged in accordance with at least some embodiments of the present disclosure. In one example configuration, computing device 1700 may include one or more processors 1710 and system memory 1720. A memory bus 1730 may be used for communicating between the processor 1710 and the system memory 1720.

Depending on the desired configuration, processor 1710 may be of any type including but not limited to a microprocessor (μP), a microcontroller (μC), a digital signal processor (DSP), or any combination thereof. Processor 1710 may include one or more levels of caching, such as a level one cache 1711 and a level two cache 1712, a processor core 1713, and registers 1714. The processor core 1713 may include an arithmetic logic unit (ALU), a floating point unit (FPU), a digital signal processing core (DSP Core), or any combination thereof. A memory controller 1715 may also be used with the processor 1710, or in some implementations the memory controller 1715 may be an internal part of the processor 1710.

Depending on the desired configuration, the system memory 1720 may be of any type including but not limited to volatile memory (such as RAM), non-volatile memory (such as ROM, flash memory, etc.) or any combination thereof. System memory 1720 may include an operating system 1721, one or more applications 1722, and program data 1724. Application 1722 may include ECG grouping algorithm 1723 that is arranged to perform the functions as described herein including the functional blocks and/or actions described. Program Data 1724 may include, among other information described, pattern matching criterion (e.g., ECG data, acceleration data, posture data, and/or QRS data) 1725 for use with the ECG grouping algorithm 1723. In some example embodiments, application 1722 may be arranged to operate with program data 1724 on an operating system 1721 such that implementations of EGM having grouping capabilities may be provided as described herein. For example, apparatus described in the present disclosure may comprise all or a portion of computing device 1700 and be capable of performing all or a portion of application 1722 such that facilitating grouping of ECG data as described herein. This described basic configuration is illustrated in FIG. 17 by those components within dashed line 1701.

Computing device 1700 may have additional features or functionality, and additional interfaces to facilitate communications between the basic configuration 1701 and any required devices and interfaces. For example, a bus/interface controller 1740 may be used to facilitate communications between the basic configuration 1701 and one or more data storage devices 1750 via a storage interface bus 1741. The data storage devices 1750 may be removable storage devices 1751, non-removable storage devices 1752, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 1720, removable storage 1751 and non-removable storage 1152 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 1700. Any such computer storage media may be part of device 1700.

Computing device 1700 may also include an interface bus 1742 for facilitating communication from various interface devices (e.g., output interfaces, peripheral interfaces, and communication interfaces) to the basic configuration 1701 via the bus/interface controller 1740. Example output interfaces 1760 may include a graphics processing unit 1761 and an audio processing unit 1762, which may be configured to communicate to various external devices such as a display or speakers via one or more A/V ports 1763. Example peripheral interfaces 1760 may include a serial interface controller 1771 or a parallel interface controller 1772, which may be configured to communicate with external devices such as input devices (e.g., keyboard, mouse, pen, voice input device, touch input device, etc.) or other peripheral devices (e.g., printer, scanner, etc.) via one or more I/O ports 1773. An example communication interface 1780 includes a network controller 1781, which may be arranged to facilitate communications with one or more other computing devices 1790 over a network communication via one or more communication ports 1782. A communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media as used herein may include both storage media and communication media.

Computing device 1700 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 1700 may also be implemented as a personal computer including both laptop computer and non-laptop computer configurations. In addition, computing device 1700 may be implemented as part of a wireless base station or other wireless system or device.

It should be appreciated after review of this disclosure that it is contemplated within the scope and spirit of the present disclosure that the claimed subject matter may include a wide variety of healthcare devices. Accordingly, the claimed subject matter is not limited in these respects.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussion utilizing terms such as “processing,” “computing,” “calculating,” “determining” or the like refer to actions or processes of a computing device that manipulates or transforms data represented as physical electronic or magnetic quantities within memories, registers, or other information storage devices, transmission devices, or display devices of the computing device.

Claimed subject matter is not limited in scope to the particular implementations described herein. For example, some implementations may be in hardware, such as those employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon that, when executed by a computing device such as a computing system, computing platform, or other system, for example, may result in execution of a processor in accordance with claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skilled in the art in light of this disclosure. In addition, those skilled in the art will appreciate that the mechanisms of the subject matter described herein are capable of being distributed as a product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity, control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular examples disclosed, but that such claimed subject matter also may include all implementations falling within the scope of the appended claims, and equivalents thereof.

What is claimed:

1. A wearable medical device (WMD) system comprising:
a plurality of electrodes, the plurality of electrodes adapted to be in contact with a person using the WMD system and to receive electrical signals from the person;
a processor communicatively coupled with the plurality of electrodes, the processor configured to analyze a plurality of electrocardiogram (ECG) signals from the received electrical signals from the person, the analysis to determine a plurality of ECG signals indicative of an arrhythmia of a heart of the person;
a storage medium communicatively coupled with the processor, the storage medium configured to store one or more episodes comprising the plurality of ECG signals determined to indicative of an arrhythmia; and
an ECG grouping module (EGM) configured to receive the stored one or more episodes, the EGM configured to:
analyze the one or more episodes to determine which of the one or more episodes meet at least one pattern matching criterion,
associate into a group the one or more episodes that meet at least one pattern matching criterion, and
cause the group to be displayed in response to a selection by a user involved with medical treatment of the person.

2. The WMD system of claim 1, wherein the EGM is included in a personal communication device, the personal communication device being communicatively coupled wirelessly with the processor.

3. The WMD system of claim 1, wherein the EGM is included in a server, the server being communicatively coupled with the processor via a network.

4. The WMD system of claim 3 further comprising a personal communication device, wherein the network comprises the personal communication device being communicatively coupled wirelessly with the processor.

5. The WMD system of claim 1, wherein the WMD system comprises a wearable cardioverter defibrillator (WCD).

6. The WMD system of claim 1, wherein the at least pattern matching criterion comprises an indication of noise included in the plurality of ECG signals.

7. The WMD system of claim 1, wherein the at least one pattern matching criterion comprises an acceleration criterion.

8. The WMD system of claim 1, wherein the at least one pattern matching criterion comprises a QRS complex criterion.

9. A method performed by an electrocardiogram (ECG) grouping module (EGM), the method comprising:
receiving a plurality of ECG signals of a person wearing a wearable medical device (WMD) via a plurality of electrodes adapted to be in contact with the person;
determining a plurality of ECG signals indicative of an arrhythmia of a heart of the person from the plurality of received ECG signals;
storing one or more episodes of the determined plurality of ECG signals indicative of an arrhythmia;
determining which of the stored one or more episodes meet at least one pattern matching criterion;
associating into a group the determined one or more episodes that meet the at least one pattern matching criterion; and
displaying the group in response to a selection by a user involved with the medical treatment of the person.

10. The method of claim 9, wherein the EGM is included in a personal communication device, the personal communication device being communicatively coupled wirelessly with the WMD.

11. The method of claim 9, wherein the EGM is included in a server, the server being communicatively coupled with the WMD via a network.

12. The method of claim 11 further comprising a personal communication device, wherein the network comprises the personal communication device being communicatively coupled wirelessly with the WMD.

13. The method of claim 9, wherein the WMD comprises a wearable cardioverter defibrillator (WCD).

14. The method of claim 9, wherein the at least one pattern matching criterion comprises an acceleration criterion.

15. The method of claim 9, wherein the at least one pattern matching criterion comprises a QRS complex criterion.

16. A wearable cardioverter defibrillator (WCD) system comprising:
a plurality of electrodes, the plurality of electrodes adapted to be in contact with a person using the WCD system and to receive electrical signals from the person;
two therapy electrodes, the two therapy electrodes adapted to be electrical in contact with the person using the WCD system and to provide a therapeutic shock to the person;
a processor communicatively coupled with the plurality of electrodes, the processor configured to analyze a plurality of electrocardiogram (ECG) signals from the received electrical signals from the person, the analysis to determine a plurality of ECG signals indicative of an arrhythmia of a heart of the person;
a storage medium communicatively coupled with the processor, the storage medium configured to store one or more episodes comprising the plurality of ECG signals determined to be indicative of an arrhythmia; and
an ECG grouping module (EGM) configured to receive the one or more episodes determined to be indicative of an arrhythmia, the EGM configured to:
analyze the one or more episodes to determine which of the one or more episodes meet at least one pattern matching criterion,
associate into a group the one or more episodes that meet the at least one pattern matching criterion, and
cause the group to be displayed in response to a selection by a user.

17. The WCD system of claim 16, wherein the EGM is included in a personal communication device, the personal communication device being communicatively coupled wirelessly with the processor.

18. The WCD system of claim 16, wherein the EGM is included in a server, the server being communicatively coupled with the processor via network.

19. The WCD system of claim 18 further comprising a personal communication device, wherein the network comprises the personal communication device being communicatively coupled wirelessly with the processor.
